(19) United States
(12) Patent Application Publication
(10) Pub. No.: US 2004/0043352 A1
(43) Pub. Date: Mar. 4, 2004

(54) Magnified dental mirror

(76) Inventor: Sybil Rose Kline, Aptos, CA (US)

Correspondence Address: Sybil Kline, 6308 Imperial Ct., Aptos, CA, 95003, US

(21) Appl. No.: 10/231,593

(22) Filed: Aug. 30, 2002

(51) Int. Cl. A61B 1/24

(52) U.S. Cl. 433/30

(57) ABSTRACT

A dental mirror comprised of a stem 5 to grasp, a head 4 tto mount a mirror, and a magnified mirror 3 that enlarges the reflected image. The magnified mirror 3 is inserted in the head 4 of a stem 5. The mirror could be magnified to any power and be placed in a stem of any design. The preferred embodiment of the magnified dental mirror would use the highest power magnification on the mirror that could focus at short range, and a stem with ridges to provide a no slip grip and ease in maneuvering the mirror around the mouth.

### CROSS-REFERENCE TO RELATED

Disclosure Document no. 479215; Sep. 5, 2000

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to a dental instrument, particularly to an improvement in the dental mirror.

Dental professionals commonly use a dental mirror in order to examine and treat a patients teeth and gum tissue. Dental mirrors are also used by the general consumer for self-examination of their teeth and gum tissue.

These dental mirrors have been made with a holder for the mirror attached to a long handle. The mirror is placed within the holder so that when the mirror is placed in a mouth, it can reflect the teeth and inside of the mouth. The mirrors used for these instruments have reflected a true representation of the teeth. Teeth are relatively small and have hard to see surfaces. Mirrors with no magnification can only reflect the actual size of the tooth and any imperfection on the tooth surface. Beginning stages of tooth decay and other dental problems can be small and difficult to see. Also, small teeth and teeth toward the back of the mouth are difficult to see. By using a magnified dental mirror the imperfection on the tooth can be enlarged and easier to diagnose and treat carefully and completely. Also hygienists who need to see areas of the tooth under the gum line and between the teeth which are very difficult to see with a actual size reflection mirror can better see these difficult to see areas by magnifying the reflected image.

2. Prior Art

Dental practitioners have long been interested in better ways to view teeth and gums of patients. Some dental surfaces are easily viewed but others require special instruments to see.

One of the first, and most commonly used instruments for viewing dental surfaces is the dental mirror. Such an instrument has the disadvantage of viewing only the actual size of the tooth surface. Small areas of decay, plaque, or other potentially harmful matter on a tooth's surface are very difficult to detect with a mirror that reflects the actual size of the tooth surface.

A dentist also needs to be able to clearly view any area suspected of damage to a tooths surface in order to accurately diagnose the problem and devise the treatment options. The small size of the tooth surface and the small indications of early signs of harm to a tooth are difficult to detect and view in detail with only an actual size reflecting mirror.

Dental mirrors are also used for treating teeth and cleaning the surfaces of teeth. Effective treatment requires that harmful matter to a tooth's surface is completely repaired or removeed in order for a tooth to be restored to a healthy state. It is necessary to clearly and completely view the damaged area of a tooth in order to provide quality care to the tooth.

A dentist also needs be able to clearly view a damaged area during treatment in order to apply a restoration that will be effectively restore the surface of a tooth. They need to be able to see that the harmful matter is removed from the tooth surface and that a restoration covers all vulnerable areas of a tooth surface and creates a smooth surface with the natural tooth. A dental mirror that reflects the actual size of the tooth in limited in its ability to reveal the details of a tooth's surface that requires treatment.

Other methods of viewing surfaces in the human mouth are available but have disadavantages. Binocular scopes are used by dentists, which are devices to wear over spectacles that magnify the view of the tooth. The disadvantages to these scopes are that they restrict the movement of the dentist and that they do not provide direct magnification to tooth surfaces that are hidden from direct view.

Another method of viewing surfaces in the human mouth is the intra-oral camera. This method projects the image of a tooth's surface onto a screeen outside the patient's mouth. It has the serious disadvantage of not being useful during treatment when a dentist needs to directly view the tooth surface.

### BACKGROUND OF THE INVENTION—OBJECTS AND ADVANTAGES

Several objects and advantages of the present invention are:

(a) to provide a method for enlarging the reflection of the surface of a tooth or gum tissue with a hand held mirror so that a dentist or hygienist can better dettect, diagnose, or treat, a tooth surface that may need treatment

(b) to provide a method for magnifying a tooth surface that does not restrict the body movement of the dental professional.

(c) to provide a method for directly magnifying the side of the tooth surface that is hidden from the dentist

(d) to provide a dental hygienist a method for detecting matter on a tooth's surface in order to completely remove harmful substances on a tooth surface SUMMARY

(e) to provide doubled magnification when used in combination with a dentists binocular scopes

In summary, the invention described here is a dental mirror with a magnifying mirror instead of a mirror that simply reflects the actual size of the tooth. Magnification of the mirror has many advantages over the prior art. It is a means to see in more detail the surface of a tooth so that a dental professional may diagnose and treat the teeth more effectively.

### DRAWINGS—FIGURES

FIGS. 1A-1B show a dental mirror with a magnified mirror, holder, and stem.

FIG. 2 shows a side view of the dental mirror.

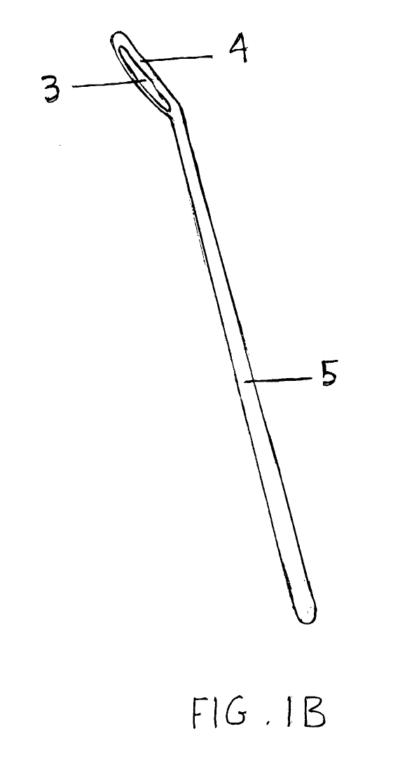

### DRAWINGS—REFERENCE NUMERALS

3 magnified mirror

4 head

5 stem

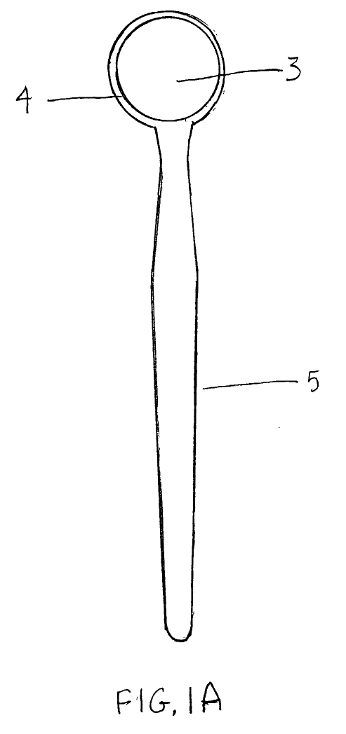

### DETAILED DESCRIPTION—FIGS. 1A AND 1B—PREFERRED EMBODIMENT

A preferred embodiment of the magnified dental mirror is illustrated in FIG. 1A (front view) patent application for Sybil R. Kline for the “Magnified Dental Mirror” and FIG. 1B (side view). FIG. 1A shows the stem 5, an elongated handle at the proximal end which is shaped into a head 4 for a mirror at the distal end, and a magnified mirror 3 placed into the head 4. This provides a dental mirror that is easy to grip and use. The stem 5 portion of the magnified dental mirror is narrower than the head 4 to provide a configuration easier to maneuver around the teeth and gums in the mouth, and easier manipulation by the user.

In the embodiment shown in FIG. 1B, the stem portion 5 is preferably on the order of ⅜ to ½ inch wide, and the length including both the head 4 and the stem 5 is approximately 5½ inches. These dimensions may be modified as required to suit individual preferences or to provide a smaller instrument, such as for the use on a child. Additionally, the shape of the stem portion 5 may be varied from that shown, such as by adding a ridges to the central portion of the stem to create surface that is easy to grip and manipulate the instrument.

The head portion 4 is wider than the stem portion 5. It serves to provide an area to mount the magnified mirror. The head 4 may be made circular or in another shape that is compatible with the shape of the mouth.

Conventional dental mirrors use circular glass mirrors which are fabricated by a drilling operation using a hole saw to cut individual mirrors out of a larger mirrored piece of glass. The magnified dental mirror can be produced similarly, by using magnified mirrored piece of glass. The magnification could be of any power but preferably of the highest power possible to clearly focus and enlarge the reflected image of the tooth or other mouth surface.

There has been described a new invention of a magnified dental mirror. The advantages of the present invention have been explained with reference to the embodiments above. Modifications of these embodiments will be made in applying the invention to different situations. The present invention should not be limited by the embodiments described above, but rather the scope of the invention should be interpreted in accordance with the claims.

I claim:

1. A hand-held dental instrument comprising:
a. a magnified mirror that will enlarge its reflection
b. an elongated handle, one portion of said handle being shaped for mounting the said magnified mirror, whereby said handle provides a means for grasping the instrument and a means for maneuvering the said mirror around the teeth and other parts of the mouth.
